(19) United States
(12) Patent Application Publication
(10) Pub. No.: US 2003/0134433 A1
(43) Pub. Date: Jul. 17, 2003

(54) Electronic sensing of chemical and biological agents using functionalized nanostructures

(75) Inventors: Jean Christophe Gabriel, Pinole, CA (US); George Gruner, Los Angeles, CA (US); Philip Collins, Irvine, CA (US); Basil Swanson, Los Alamos, NM (US); Fred Wudl, Los Angeles, CA (US)

Correspondence Address: NANOMIX, INC., 5980 HORTON ST. 6TH FLOOR, EMERYVILLE, CA, 94608, US

(73) Assignee: Nanomix, Inc., Emeryville, CA (US)

(21) Appl. No.: 10/345,783

(22) Filed: Jan. 16, 2003

Related U.S. Application Data
(60) Provisional application No. 60/349,670, filed on Jan. 16, 2002.

(51) Int. Cl. C12M 1/34; G01N 33/543

(52) U.S. Cl. 436/518; 435/287.2

(57) ABSTRACT

This invention provides for an apparatus and a method for detecting the presence of pathogenic agents with sensors containing functionalized nanostructures integrated into circuits on silicon chips. The nanostructures are functionalized with molecular transducers that recognize and bind targeted analytes which are diagnostic of the pathogenic agent of interest. The molecular transducer includes a receptor portion, which binds the analyte, and an anchor portion that attaches to the nanostructure. Upon binding of the analyte, a change in molecular configuration represented by the newly formed receptor-analyte complex creates a force that is transmitted to the nanostructure via the anchor portion of the transducer. The effect of the force transmitted to the nanostructure is to alter its conductivity. The change in conductivity of the nanotube thus represents a signal that indicates the presence of the pathogenic agent of interest.

### DOMESTIC PRIORITY CLAIM

The priority of U.S. Provisional Application No. 60/349,670, filed Jan. 16, 2002, is claimed.

### FIELD OF THE INVENTION

The invention relates generally to the sensing of biological agents and, more specifically, to using nano-electronic circuits as transducers to convert and to amplify signals produced by the biological agents and methods for making such nano-electronic circuits.

### BACKGROUND

There is intense interest in the development of small solid-state sensors that are capable of rapidly detecting markers for human bacterial pathogens, including biological threat agents. Conventional detection methods are generally based on the use of biological reagents for specific recognition of biological targets, including the mutual recognition that complimentary oligonucleotides have for each other, and the recognition of antigens by specific antibodies. In general, recognition and binding events are coupled with some form of optical detection that relays a signal indicating detection of the target of interest.

DNA recognition techniques are quite accurate, as they are based on the principle of a DNA sequence signature that is unique to each bacterial species and strain. Sections of the DNA are tagged with markers that can be detected using fluorescent methods. However, as there is only a very small amount of DNA material in a cell or in any given sample, the technique relies on the polymerase chain reaction (PCR) technology to increase the quantity of the sample material to a level that allows detection by fluorescent techniques. The combination of these techniques is expensive, complex, and time consuming; DNA recognition techniques can require a day for identifying a typical bacterial species, and three days for identifying a typical strain.

Human bacterial pathogens belong to either the gram-positive or the gram-negative Proteobacteria divisions; within these divisions, the pathogens are classified according to Genus, Species, and Strain. A large number of specific antibodies have been identified that recognize and bind to particular species of bacterial pathogens, and thus immunoassay techniques use these specific antibodies to detect particular bacterial pathogen species. Immunoassay techniques are generally less discriminating, but faster and more economical than DNA recognition techniques. Their accuracy may be limited by the specificity of the antibody-antigen combination, more particularly by cross-reactivity, i.e., the degree to which the antibody binds to species other than the target of interest. Fluorescent detection methods are very sensitive, but the large, sensitive fluorescence detectors of laboratory systems are costly.

Rapid screening tests have also been developed that utilize colorimetric detection, wherein the changing of color within an assay solution indicates detection of a target of interest. These systems are generally more economical, but less sensitive than fluorescent methods, as the signal involves not only change in wave length, but variation in intensity of color. Such tests can be read by the human eye, or with the aid of an optical reader.

Recently, detection techniques involving nanostructures (such as nanotubes or nanowires) as sensors have been developed. A nanostructure is used as a constricted path that conductively spans the gap between two electrodes. An electrical current is forced to pass through this constriction, and the current flow is then measured across the electrodes. In another architecture, a field effect transistor (FET) is created, where the conductivity across the nanotube, or nanowire, is controlled by the application of a gate voltage. A change in the electronic properties of the nanostructured electrical pathway in either or these alternative architectures affects the electronic response measurably. In particular, if a molecule binds to the surface of the nanostructured electrical element, it can significantly change the flow of current across the constriction, and such a change in the electronic properties can be used to detect the presence of the molecule. The use of nanostructures for electronic detection of the presence of gases has been described by Bradley et al. (Physical Review Letters 85, 412-21) and by Collins et al. (Science 287, 1801). The detection of gases is based on the observation that the electrical properties of carbon nanotubes, including the resistance, thermoelectric power, and local density of states, change considerably when the nanotubes are exposed to oxygen.

Balavoine et al. (Angewandte Chemie Int. Ed. 111, p. 2036) have shown that it is possible to organize proteins on the surface of a carbon nanotube. These authors suggest that nanotubes decorated with biological macromolecules could form the basis for the development of new biosensors and bioelectronic nanomaterials, taking advantage of the specific biomolecular recognition properties associated with the bound macromolecules. For such purposes, the specific recognition protein would need to be densely packed on the surface of the carbon nanotubes and the protein would need to be functional with regard to its ability to recognize and bind with the target of interest.

Chen et al. (J. Am. Chem. Soc. 123, p. 3838) have reported a general approach to non-covalent functionalization of the sidewalls of single-walled carbon nanotubes, and the subsequent immobilization of various biological molecules onto the nanotubes, that yields a high degree of control and specificity. Cui et al. (Science vol. 293, p. 5533) have taken this approach a step further and demonstrated that it is possible to achieve very fast protein detection using silicon nanowires (SiNW) and the model provided by the well-characterized ligand—receptor binding relationship between biotin and streptavidin. These authors functionalized the surface of a SiNW with biotin and detected a change in the conductivity of these functionalized SiNWs after exposure to streptavidin. The change in conductance of the SiNWs upon exposure to streptavidin was not a general feature of the SiNWs, but was dependent on the functionalization with biotin. The sensitivity of this device was reported to be at the level of 10 ppM (part per million).

Song et al. (U.S. Pat. No. 6,297,059 B1) have described an optical biosensor that includes a substrate having a lipid bilayer membrane thereon. A recognition molecule, capable of binding a target biomolecule, is disposed within the membrane. The recognition molecule includes a fluorescent label which changes in fluorescence in response to binding between the recognition molecule and the target biomolecule. Distance-dependent fluorescence self-quenching and/or fluorescence energy transfer are used as optical signal transduction mechanisms to detect molecule-target biomolecule interactions.

There is a need for improved processes and materials that are capable of sensing analytes that represent targeted bio-molecules, or groups of related molecules, with high specificity and with high sensitivity, particularly where these bio-molecules are associated with, or representative of, or diagnostic of human pathogenic organisms. Preferably, sensing devices have fast detection response and will be amenable to being configured into portable and robust packages. There are many applications where a robust, sensitive, fast, accurate and multiply-specific architecture for recognition of biological agents, which utilizes simple electronic signal amplification technology and can be used under a wide variety of conditions, would be useful.

### SUMMARY

According to the invention, nano-sensors are presented that are capable of detecting biomolecular analytes, those particular to a human pathogen, by using an electronic nanocircuit as a transducer and amplifier. Such biomolecular analytes include DNA sequences, RNA sequences, or proteins, or any molecule whose presence is diagnostic of a particular human pathogenic organism. Such pathogens include various species of bacteria, protozoa, fungi, viruses and prions. A nanocircuit contains one or more nanostructures, examples of which include nanofibers, single-walled nanotubes, multi-walled nanotubes, nanocages, nanococoons, nanohorns, nanotopes, nanotori, nanorods, nanowires and other configurations of fullerene-like molecules constructed of carbon and/or other light elements. The nanostructures are integrated into a device that can measure changes in electrical behavior along the nanostructured conductive path.

In some embodiments, the inventive device includes a semiconductor chip with metal lines or electrodes. A nanostructure spans the gap between two electrodes, thus serving as a molecular wire electrically coupled to the electrodes through which electric current passes, as it travels from one electrode to the other. Changes in properties of the nanostructure dramatically affect features of its electrical performance, which can be measured by applying a low voltage across the device. The control circuitry of the sensor can be formed on the same semiconductor chip. The use of a semiconductor chip as the technology platform allows the utilization of standard integrated circuit fabrication technology.

In some embodiments, a biologically and chemically protective coating or membrane layer surrounds the electronic nanocircuit. One of the functions of the coating layer is to diminish, inhibit, or prevent the non-specific binding or interaction of non-targeted analytes with the nanostructure. The protective coating can include a surfactant or lipid monolayer, bilayer, multilayer, hybrid bilayer, a self-assembled monolayer of biological or non-biological material, a polymer layer (such as PEG polyethylene glycol), a micellar layer, or other macromolecular assemblies composed of amphipathic molecules. The biologically and chemically protective coating or layer can contain both hydrophobic and hydrophilic elements, as well as molecular transducers or ion channels, which respond selectively to target molecules.

In some embodiments, nanostructures are clustered into an integrated array on a single semiconductor chip. Subgroups of nanostructures are each functionalized for the detection of specific biological target molecules. In such a configuration, the range of pathogens that can be detected can be easily expanded. In some embodiments a library of pathogen specific sensors is created, which can be used as part of a larger sensor system. In still other embodiments, the sensor device is integrated into a larger instrumentation package that includes sample handling, transmission of data to remote sites, alarm capabilities, and computer control of operations.

### BRIEF DESCRIPTION OF THE DRAWINGS

FIG. 1 schematically illustrates a biomolecular nano-sensor in accordance with some embodiments of the invention.

FIGS. 2A-F schematically illustrate embodiments of the biomolecular nano-sensor device.

FIG. 3 schematically illustrates a biomolecular transducer containing an anchor, a tether, and a receptor segment.

FIG. 4A schematically illustrates a functionalized nanostructure nano-sensor device with target biomolecules absent.

FIG. 4B schematically illustrates a functionalized nanostructure nano-sensor device with target biomolecules bound to receptor sites.

FIG. 5A schematically illustrates a sequence of steps for forming a biomolecular sensor according to some embodiments of the invention.

FIG. 5B schematically illustrates a sequence of steps for forming a biomolecular sensor according to some embodiments of the invention.

### DETAILED DESCRIPTION

The Biological Nanosensor

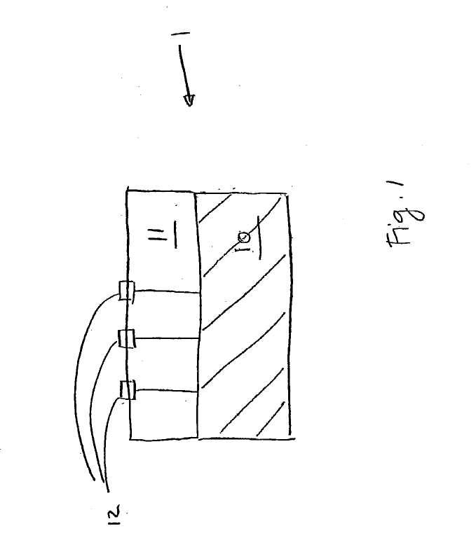

FIG. 1 illustrates a biological nano-sensor 1 in accordance with some embodiments of the invention. Such a nano-sensor is defined as nanostructure-based sensor that senses the presence of biological agents by way of detecting a biological molecule derived from such an agent. Nanostructures include any one or any subset of nanostructures that include nanofibers, single-walled nanotubes, multi-walled nanotubes, nanocages, nanococoons, nanohorns, nanotopes, nanotori, nanorods, nanowires and/or other fullerene-like molecules, as well as other extended molecules such as polymers, dendrimers, and organometallics. A fullerene is a closed, convex cage molecule constructed from carbon, containing only hexagonal and pentagonal faces, whose prototype is the C₆₀ buckyball. By “fullerene-like” is meant the broad molecular family of variations thereon that retain the basic molecular and electronic features of the prototype. Variants include both closed, cage-like forms and extended linear forms, such as those listed above, as well as those with other atoms included, and with various features considered to be defects or imperfections compared to the C₆₀ fullerene prototype. Examples of atoms other than carbon known to be included in such variant fullerenes include boron, boron nitride, and carbon boron nitride, silicon, germanium, gallium nitride, zinc oxide, indium phosphide, molybdenum disulphide, and silver.

Such a biological nano-sensor detects the presence of a biological agent through a specific binding event that is transduced by molecular mechanisms as detailed below, which create a characteristic electrical signal. As depicted in FIG. 1, nano-sensor 1 includes a nanostructure 10 that is capable of changing its electrical properties when coupled to a targeted molecule. Nanostructure 10 is insulated from its local environment, wherein sensing interactions occur, by an insulating coating layer 11. The coating layer 11 prevents non-specific binding or interaction of non-targeted molecules with nanostructure 10, and which could thereby alter the conductivity of the nanotube. One or more molecular transducers 12, which represent a form of functionalization that is responsive to specific target molecules, are positioned so as to extend through coating layer 11. The molecular transducers 12 are coupled to nanostructure 10. When a molecular transducer 12 interacts with a targeted molecule near the surface of coating layer 11, it transmits a signal representing the sensing of the targeted molecule to the nanostructure 10 through the coupling between the molecular transducer 12 and the nanostructure 10. Full functionalization of the nanostructure is thus accomplished by the coating and the molecular transducer together. The coating has a role in markedly decreasing the signal noise that would accompany nanotube interaction with non-target analyte; the transducer has a role in positively selecting the target analyte from within a sample that contains both the targeted analyte and spurious, non-targeted compounds.

The Biological Nanosensor Integrated into a Semiconductor Chip

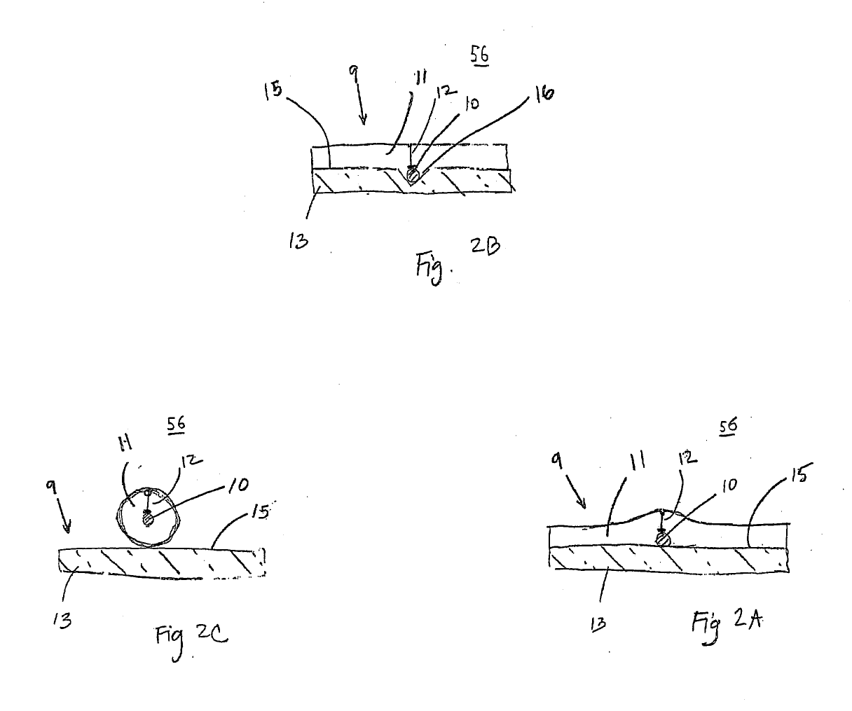

FIGS. 2A-F illustrate embodiments of the biological nano-sensor device 9, wherein functionalized nanostructure 10 is integrated into a semiconductor chip. The semiconductor chip contains a substrate 13 and electrodes (not shown in this figure). Control circuitry (not shown in this figure) for nano-sensor device 9 can be provided on the same semiconductor chip. The substrate 13 can be made of any of a variety of materials or layers of materials consistent with the art of semiconductor manufacturing. The substrate 13 can include semiconducting material(s), such as silicon, III-V compounds, II-VI compounds, or a combination of one or more Group IV elements with any of these. Alternatively or in combination with semiconducting materials, the substrate 13 can include insulating material(s), such as alumina, silicon dioxide, and/or quartz. The use of a semiconductor chip as the technology platform allows the utilization of standard integrated circuit fabrication technology.

In FIGS. 2A-F, nanostructure 10 is disposed over a substrate 13. The surface 15 of the substrate 13, which is facing the nanostructure 10, can include silicon, silicon dioxide, or any other material consistent with semiconductor manufacture. The nanostructure 10 configurations can include any one or any subset of nanostructures that include nanofibers, single-walled nanotubes, multi-walled nanotubes, nanocages, nanococoons, nanohorns, nanotopes, nanotori, nanorods, nanowires and/or a fullerene-like molecules, as well as other extended molecules such as polymers, dendrimers, and/or organometallics. In some embodiments, the nanostructure 10 can be formed from any element or combination of elements known to form nanostructures, such as carbon, boron nitride, or boron. The particular embodiments of FIGS. 2A-F show a nanostructure 10 as a nanotube. The cross sectional view of FIGS. 2A-C is cut near the center of the nano-sensor device 9. The cross-sectionally depicted nanotube 10 extends upward and downward with respect to the plane of the page and makes contact with one or more electrodes both above and below the page (not shown). Combinations of the embodiments shown in FIGS. 2A-F are also possible.

FIG. 2A illustrates a cross sectional view of an embodiment, where the nanostructure 10 is positioned adjacent to the surface 15 of the substrate 13. A biologically and/or chemically protective layer 11 is disposed over the nanostructure 10 and substrate surface 15, covering and insulating the nanostructure from the local environment 56 into which control and test samples are received.

FIG. 2B illustrates a cross sectional view of an embodiment, wherein the nanostructure 10 lies in a groove 16, formed on or within the surface 15 of the substrate 13. The protective layer 11 is disposed substantially over the nanostructure 10 and substrate surface 15, covering at least a portion of the nanostructure and insulating it from the environment 56.

FIG. 2C illustrates a cross sectional view of an embodiment wherein the nanostructure 10 is positioned adjacent to the surface 15 of the substrate 13. The protective layer 11 is disposed substantially or at least partially around the surface of the nanostructure 10. Although not shown in this view, the protective layer 11 can also be disposed along the surface 15 of the substrate 13.

FIG. 2D illustrates a side view of an embodiment wherein the nanostructure 10 is positioned between electrodes 32, suspended within the environment 56, above substrate 13. Suspending the nanostructure 10 in such a manner separates it from impurities and other uncontrolled or unwanted deposits that may be on the surface 15 of substrate 13. Electrodes 32 are positioned adjacent to substrate 13. Insulating layers 21 overlie at least portions of electrodes 32 and provide electrical and/or chemical insulation for them. Protective layer 11 is disposed adjacent to both the insulating layers 21 and the nanostructure 10, and at least partially insulates nanostructure 10 from its environment 56. Layer 11 may also at least partially insulate layers 21 from the environment. Insulating layers 21 define an opening 23. In the opening 23 one or more molecular transducers 12 are disposed within protective layer 11 so as to extend beyond it and into the local sensing environment 56, providing communication between nanostructure and the local sensing environment.

FIG. 2E illustrates a side view of an embodiment, where the nanostructure 10 is positioned between electrodes 32, suspended within the environment 56. Positioning nanostructure 10 in such a manner separates the nanostructure from the impurities and other uncontrolled or unwanted deposits that may be on the surface 15 of substrate 13. Electrodes 32 positioned adjacent to substrate 13. Insulating layers 21 positioned adjacent to electrodes 32 and provide electrical and chemical insulation for them. Insulating layers 21 define an opening 23. Protective layer 11 is disposed in opening 23, positioned adjacent to nanostructure 10, and insulates it from the environment 56. Dispersed within the protective layer 11 are one or more molecular transducers 12, providing communication between nanostructure 10 and the environment 56.

FIG. 2F illustrates a-planar view of a nano-sensor chip 27, formed as an array of nano-sensor devices disposed on a substrate 13. On substrate 13 an array of pairs of electrodes 32-1,1, . . . , 32-N,M are formed. An insulating layer 21 positioned adjacent to electrodes 32 in some embodiments in a manner illustrated in FIG. 2D, and in some embodiments in a manner illustrated in FIG. 2E. Insulating layer 21 defines an array of openings 23-1,1, . . . , 23-N,M. Nanostructures 10-1,1, . . . , 10-N,M are disposed between pairs of corresponding electrodes 32-1,1, . . . , 32-N,M. In some embodiments the protective layer 11 substantially overlies the entire nano-sensor chip 27, in other embodiments protective layer 11 overlies the nano-sensor chip piece by piece. In other embodiments, protective layer 11 is formed individually in the openings 23, forming an array of protective layers. In each embodiment, protective layer 11 insulates nanostructures 10 from the environment 56. Furthermore, an array of molecular transducers 12-1,1, . . . , 12-N,M is formed in protective layer 11 in the regions of openings 23. One or more molecular transducers 12 are disposed in the openings 23, providing communication between nanostructures 10 and the environment 56.

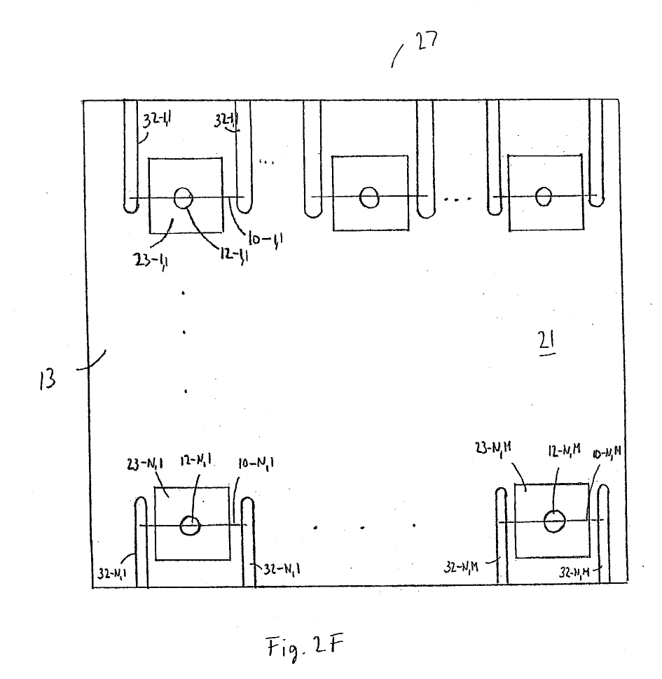

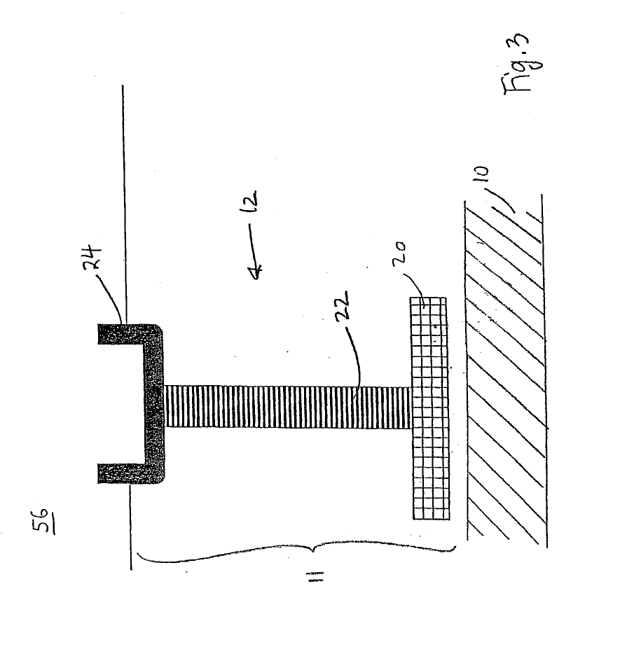

Electrodes 32 are coupled or conected to additional circuitry (FIGS. 4A, 4B), which can be positioned on nano-sensor chip 27, or off-chip, in some other part of the sensor device. The additional circuitry is capable of analyzing the changes of electrical conductance between individual pairs of electrodes, sensing the presence, or absence of the targeted molecules. In some embodiments the molecular transducers of different openings are specific to different targeted molecules. These embodiments of nano-sensor-chip 27 are capable of analyzing the presence or absence of several targeted molecules in a test sample in parallel, greatly accelerating the process of a thorough sample analysis.

As shown in FIGS. 2A-F the biologically and/or chemically protective coating 11 inhibits or prevents non-specific binding of non-targeted molecules to the nanostructure 10, thus enhancing the sensing specificity of the nano-sensor device 9. The protective coating 11 can include such forms as a phospho-lipid monolayer, a phospho-lipid bilayer, a hybrid surfactant bilayer, a surfactant multilayer, a self-assembled monolayer of biological or non-biological material, a polymer layer (such as PEG, polyethylene glycol), a micellar layer, and/or surfactants. The coating 11 can contain hydrophobic and hydrophilic ligands. In some embodiments an aqueous layer can be disposed between the nanostructure 10 and the protective layer 11. Some of these films are described by Schwartz (Surface Science Reports 27, 245-334, 1997) and by Knobler et al. (Current Opinion in Colloid & Interface Science 4, 46).

The functions of the protective layer 11 include preventing or inhibiting non-targeted molecules from coupling to the nanostructure 10, and, in some embodiments, to provide a cell membrane-like environment for targeted molecules. In some embodiments, highly flexible protective layers 11 are selected that can be bent with a small bending radius. The flexibility of protective layers 11 can be increased with co-surfactants, or with the addition of small alcohol molecules.

The process of binding of targeted biomolecular analyte and initial signal generation in response thereto is performed by molecular transducers 12, as illustrated in FIGS. 2A-F. The molecular transducer 12 extends from the outer surface of the protective layer 11 to the proximity of nanostructure 10 through the protective layer. The molecular transducer 12 is capable of sensing and binding a targeted molecule and communicating the sensing and binding of the targeted molecule to the nanostructure 10. Molecular transducers can include, for example, pathogen-specific molecules that are sensitive to human bacterial pathogens. In some embodiments, molecular transducers can include an antibody, or a portion thereof. Although only one molecular transducer 12 is illustrated in FIGS. 2A-F, in other embodiments there can be two or more molecular transducers 12 formed in each protective layer 11, and there can also be more than one type of molecular transducer formed in each protective layer. Details of the embodiments of molecular transducers are described below.

The Molecular Transducer

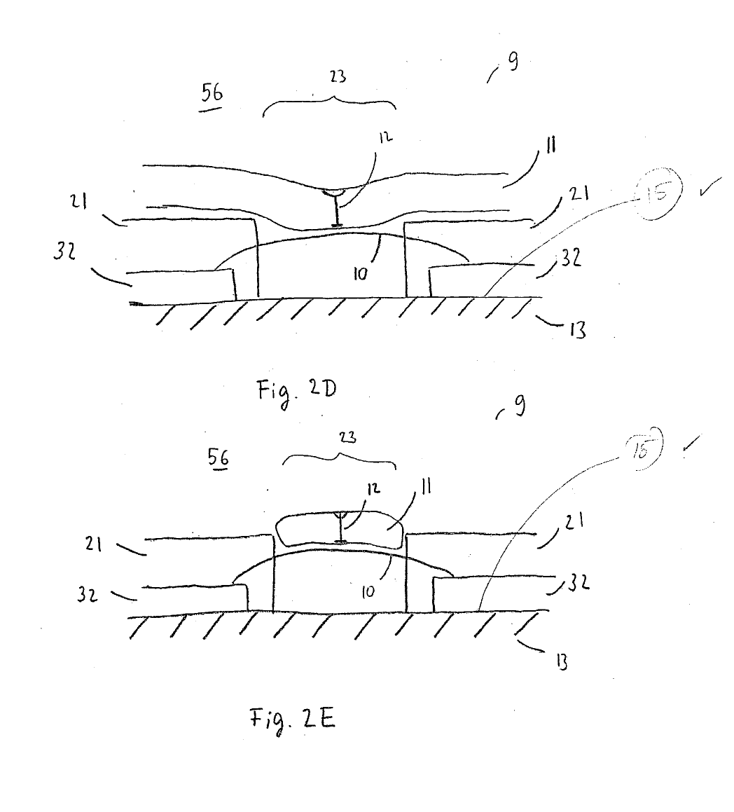

FIG. 3 illustrates some basic constituents of the molecular transducer 12, which includes an anchor molecule 20, a tether molecule 22, and a receptor molecule 24. The anchor molecule 20 portion of the transducer is attached both to the nanostructure 10 and to the tether molecule 22. The role of the anchor molecule 20 is, by way of its interaction with the nanostructure 10, to modify the conductivity properties of the nanostructure when binding between the receptor molecule portion of the transducer and a target analyte biomolecule occurs. Example anchor molecules include amine groups, polyaromatic molecules, and covalently bound groups. The anchor molecule 20 can be bound to the nanostructure 10 by covalent bonding, or non-covalent bonding. Non-covalent bonds include hydrophobic bonds, ionic bonds, and π-bond overlaps. Examples of known interactions that can modify the conductivity include those that are electrostatic in nature, as described by Kim et al. (Physical Review B vol. 64, no. 15,p. 3404), those that can be affected by a charge transfer mechanism, as described by Jhi et al. (Phys Rev. Lett. 2000, 85, 1710) and by Léonard et al. (J. Phys. Rev. Lett. 1999, 83, 5174), and those that can be due to a variation in the local structure of the nanostructure, as described by Okada et al. (Journal of the Physical Society of Japan 70, 2345), by Liu et al. (Physical Review B. 6403, no. 3), and by Hansson et al. (Physical Review B. 62, no. 11).

The molecular transducer 12 further includes tether molecule 22, which is connected both to the anchor molecule 20 and the receptor molecule 24. The tether molecule 22 extends through the biologically and chemically protective coating 11, and provides a connection between the anchor 20 near the nanostructure 10 and a receptor 24 near the surface of the protective coating. The tether molecule 22 can be configured at various angles with respect to the coated nanostructure, including being approximately perpendicular to the surface of the protective coating 11. The tether molecule 22 can contain more than one kind of functional moiety, and can include lipophilic, liposoluble, and/or hydrophobic groups, and can be branched or linear in form. Candidates for tether molecules 22 include single- or poly-saturated aliphatic chains, unsaturated aliphatic chains, and aromatic chains. The length of the tether molecule 22 can be selected to fit the thickness of the protective coating 11. In some embodiments, the tether molecule is relatively stiff, in which case a movement on one end of the tether molecule causes a similar movement on the other end of the molecule, providing a mechanism for communicating or transducing the sensing of a targeted molecule to the nanostructure 10. In other embodiments, the length of the tether molecule can be between about 6 and about 7 nanometers. In still other embodiments, the anchoring and tethering functions are embodied within a single multifunctional anchor molecule that is connected both to the nanostructure and the receptor molecule.

The molecular transducer 12 further includes a receptor molecule 24 that binds specifically to a targeted biomolecule of interest for its association with a human pathogenic organism. By biomolecule is meant any molecule of biological origin, such as lipids, peptides, proteins, carbohydrates, polysaccharides, oligonucleotides, polynucleotides, conjugate molecules such as glycoproteins, and glycolipids, or molecules of biological intermediary metabolism. The receptor molecule 24 can be protein, such as an antibody, an enzyme, or the active site of an antibody or an enzyme, a polynucleotide, such as a DNA or an RNA sequence, a carbohydrate, a cyclodextrin, a crown ether, or any other molecule capable of specifically recognizing and interacting with or binding to a biomolecule of interest, or capable of being functionalized in such a way that it recognizes or binds specifically to a biomolecule of interest. The receptor molecule 24 can also be coupled to the tether molecule 20 by way of a linker molecule (not shown).

Operation of the Biological Nanosensor

FIG. 4A shows nano-sensor device 9 according to an embodiment of the invention in which the nanostructure 10 of the nano-sensor device is a nanotube 10. The nanotube 10 is positioned over or otherwise adjacent to an insulating layer 30, such as silicon dioxide or other insulating material, which is positioned adjacent to a silicon substrate 13. The biologically and chemically protective layer 11 substantially or at least partially encapsulates the nanotube 10 thus providing it insulation from its environment.

The nanotube 10 is electrically coupled or connected to contact electrodes 32, which are electrically connected to an electrical circuit 40. The nanotube 10 has an original conductivity which determines the current conducted in response to a given applied voltage. A voltage source 42 applies a voltage to the electrodes 32 and generates a current through nanotube 10 and the generated current is read by ammeter 44. In some embodiments, the voltage source 42 can apply a voltage to the substrate 13, which then can act as an undifferentiated gate electrode for the device 9. The gate voltage can be DC, AC, or both. The current is very sensitive to the electrical conducting properties of the nanotube 10; small changes in the electrical conducting properties of the nanotube are readily detectable as a change in current flow.

A processing unit 60, which may include a computer or computational device (see FIG. 4A) may be used with any of the embodiments described herein, with appropriate software and/or hardware configured to provide near real-time and historical analysis of changes in conductivity of single sensors or arrays of sensors according to the invention. In one embodiment of the nanostructure sensing device, it is housed in an instrumentation unit which includes a computer-controlled signal control and processing unit, a sample handling device for delivery of test samples to be analyzed, and a transmitter that sends data by wire or wireless methods to remote sites. Recording devices are also included in the instrumentation, either at the site of the sensing device or at remote sites, to maintain a historical record of signal activity, and to provide immediate alarming under certain conditions. The data recording software includes a pre-set signal threshold, which if exceeded by sensing signal output, triggers an alarm system, that can be situated at either the site housing the instrumentation or at remote sites.

The protective layer 11 includes molecular transducers 12-1, 12-2, and 12-3 that are capable of sensing one or more targeted molecules. Under control conditions, in the absence of the targeted analyte biomolecule, the anchor molecules 12-1a, 12-2a, 12-3a of the molecular transducers 12-1, 12-2, 12-3 maintain an equilibrium charge transfer with the nanotube 10 and an equilibrium distance from the nanotube 10. Under non-sensing conditions, a characteristic non-sensing equilibrium current flows through the nanotube 10, as determined by the conductance of the nanotube under control conditions, and as read by the ammeter 44. The tether molecules 12-1b, 12-2b, and 12-3b provide a mechanical link between the anchor molecules 12-1a, 12-2a, and 12-3a and the receptor molecules 12-1c, 12-2c, and 12-3c. The length of the tether molecules is selected to provide a responsive connection between the anchor molecules 12-1a, 12-2a, 12-3a and the receptor molecules 12-1c, 12-2c, 12-3c. In some embodiments the length of the tether molecules is commensurate with the thickness of the protective layer 11. In the embodiment of FIG. 4A, receptor molecules 12-1c, 12-2c, 12-3c have a specificity for different targeted molecules. In other embodiments, all receptor molecules can have specificity for the same targeted molecule.

FIG. 4B illustrates the nano-sensor device 9 of FIG. 4A after biological targeted molecules have become bound to the receptor molecules 12-1c, 12-2c, 12-3c. Target molecule 46 contains more than one kind of binding site and has bound to receptor molecules 12-1c and 12-2c. Target molecule 48 contains only one kind of binding site and has bound to receptor 12-3c.

The binding of the targeted molecules from a test sample to a receptor molecule creates a receptor-ligand complex with a newly present molecular configuration that exert a force on the molecular transducers 12-1, 12-2, and 12-3. The molecular configuration of the receptor-ligand complex, or of the receptor itself, as modified by its interaction with the ligand, that gives rise to the force on the molecular transducers can be of a steric or an electrostatic nature. Such an altered configuration causes repulsion or attraction between the protective layer 11 and the targeted molecule bound to receptor molecules 12-1c, 12-2c, and 12-3c. The bound ligand molecule, or the receptor-ligand complex as a unit accommodates the steric or electrostatic force by moving with respect to protective layer, moving the receptor molecules with it. The receptor molecules, in turn, exert a force on the tether molecules 12-1b, 12-2b, and 12-3b. Finally, the tethered molecules transmit the force to the anchor molecules 12-1a, 12-2a, 12-3a, thereby perturbing them and changing the nature or magnitude of their electronic interaction with the nanotube 10. Such change in the electronic relationship between the anchor molecule and the nanotube, in turn, causes a change in the electrical conductivity of the nanotube portion of the sensor circuit.

In some embodiments, for example, the interaction between the anchor molecule 12-1a and the nanotube 10 is strong enough that the transmitted force causes a structural deformation of the nanotube, such as a localized bending. In other embodiments, the binding between the anchor molecule 12-3a and the nanotube 10 is weaker, and the force results in change in the distance between the anchor molecule 12-3a and the nanotube, thus causing a change in charge transfer between the anchor molecule and the nanotube. In some embodiments, the anchor molecule affects the nanotube by a modification of the charge transfer, by a change in the local electric field, by a change in the distortion of bonds, or by a change in the π-bond overlaps.

Any changes in the relationship between the anchor molecules 12-1a, 12-2a, 12-3a and the nanotube 10 result in a change in the electrical conduction properties of the nanotube, for example, in its electrical resistance. Given the one-dimensional nature of the electrical conduction path, even a localized increase in the electrical resistance of the nanotube 10 creates a bottleneck for the conduction path and thus causes a large increase in the overall resistance of the nanotube. A large increase is resistance causes a large decrease in the current that can be sensed by ammeter 44. Some interactions can cause a decrease in resistance, leading to an increased current that can be sensed by ammeter 44. Processing unit 60, as noted above, can be used to provide more complex analyses of analyte detection, and may include ammeter 44 or be separate from it.

In embodiments where the receptors are able to bind a specific targeted molecule with high specificity, a change in current indicates the sensing and binding of the targeted molecule. In embodiments where the receptors are sensitive to more than one type of targeted molecule, a calibration is performed to relate particular changes in the measured current to the binding of particular targeted molecules.

Forming the Biological Nanosensor

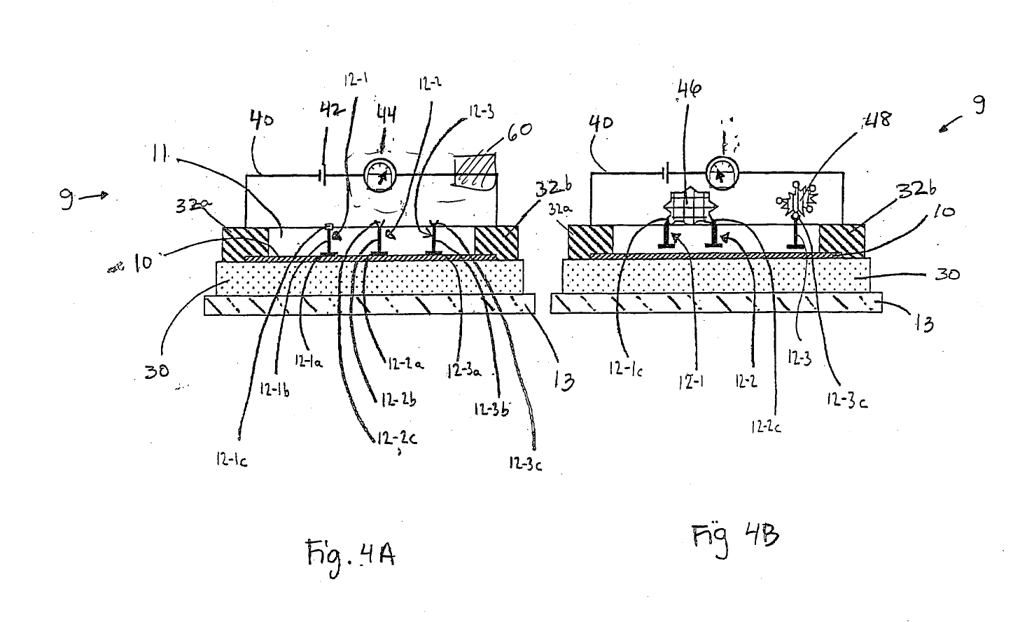

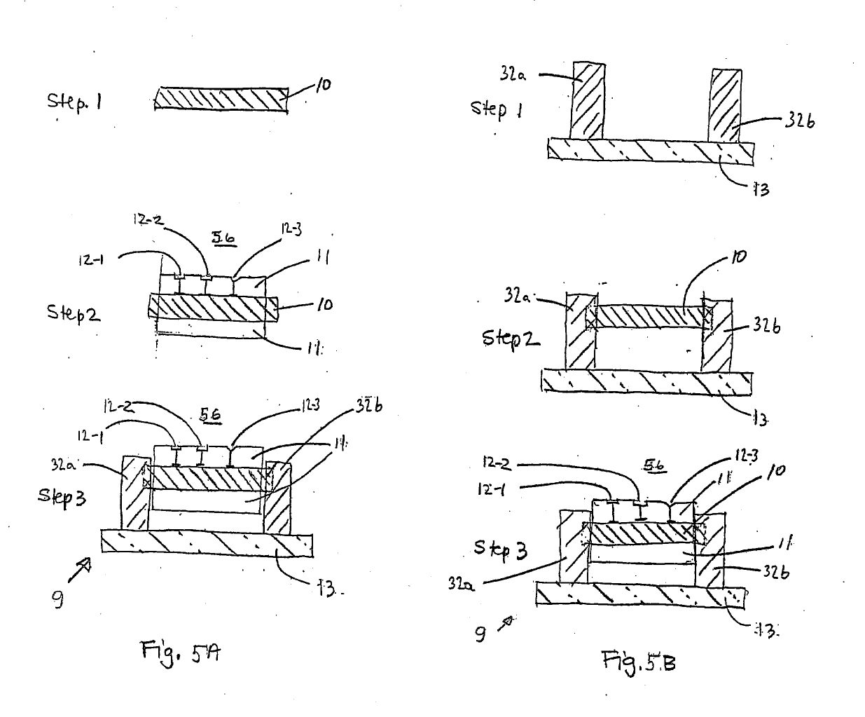

FIG. 5A illustrates schematically a sequence of steps for forming the nano-sensor device 9 according to some embodiments of the invention. In Step 1, nanostructure 10 is formed; the nanostructure may include a nanofiber, a single-walled nanotube, a multi-walled nanotube, a nanocage, a nanococoon, a nanohorn, a nanotope, a nanotorus, a nanorod, a nanowire and other extended molecules such as polymers, dendrimers, organometallics, and fullerene-like molecules, and may include any subset or combination of such nanostructures. The embodiment depicted in FIG. 5A shows a nanotube 10, which can be formed from any element or compound known to form such nanostructures, including carbon, boron nitride, and boron. Details of the formation and structure of nanostructures are described in U.S. patent application Ser. No. 10/020,392 of Bradley et al., “Hydrogen Storage in Nanostructures with Physisorption”, and U.S. patent application Ser. No. 10/020,344 of Kwon et al., “Increasing Hydrogen Adsorption of Nanostructured Storage Materials by Modifying sp² Covalent Bonds”.

In Step 2, the biologically and/or chemically protective coating, or membrane layer 11 is formed around at least portion of the nanostructure 10 as part of the nanostructure functionalization process. The protective layer 11 can include, for example, a surfactant or monolayer, bilayer, hybrid bilayer, a multilayer, a self-assembled monolayer of biological or non-biological material, a polymer layer (such as PEG polyethylene glycol), and/or a micellar layer The protective layer 11 can contain hydrophobic and hydrophilic elements. Molecular transducers 12-1, 12-2, and 12-3 are also situated within the protective layer 11. In this illustration, two types of molecular transducers are provided; one type is shown as 12-1 and 12-2, the other type is shown as 12-3. In other arrangements, there may be only one kind of molecular transducer or there may be any number of kinds of molecular transducers provided in the protective layer 11. The molecular transducers 12-1, 12-2, 12-3 provide communication through the protective layer 11 between the nanostructure 10 and the local environment 56 in which control or analyte test samples are applied. The molecular transducers contain receptors that bind to specific molecules of interest from the environment 56, as was discussed in more detail above with reference to FIGS. 3 and 4.

In Step 3, the functionalized nanostructure 10 is integrated into a semiconductor chip, containing a substrate 13 and electrodes 32a and 32b. Control circuitry (not shown) for the nano-sensor device 9 can be provided on the same substrate 13, as part of a silicon chip. The use of a silicon chip as the technology platform allows the utilization of standard integrated circuit fabrication technology.

FIG. 5B schematically illustrates a sequence of steps for forming a biological sensor according to other embodiments of the invention. In Step 1, a semiconductor chip is provided that contains a substrate 13 and electrodes 32a and 32b. Control circuitry (not shown) for the nano-sensor device 9 can be provided on the same substrate 13, as part of a semiconductor chip. The use of a silicon chip as the technology platform allows the utilization of standard integrated circuit fabrication technology.

In Step 2, a nanostructure 10 is formed above the surface of the semiconductor chip, extending from one electrode 32a to the other electrode 32b. The nanostructure 10 may include a nanofiber, a single-walled nanotube, a multi-walled nanotube, a nanocage, a nanococoon, a nanohom, a nanotope, a nanotorus, a nanorod, a nanowire and/or a fullerene-like molecule. The nanostructure contains at least one, but can also contain many different nanostructures. The embodiment of FIG. 5A shows a nanotube structure 10. The nanostructure 10 can be formed from any element known to form nanostructures, such as carbon, boron nitride, or boron.

In Step 3, a biologically and chemically protective coating or membrane layer 11 is formed around the nanostructure 10 as part of the nanostructure functionalization process. The protective layer 11 can include a surfactant or lipid monolayer, bilayer, hybrid bilayer, a self-assembled monolayer of biological or non-biological material, a polymer layer (such as PEG polyethylene glycol), and a micellar layer. The protective layer 11 can contain hydrophobic and/or hydrophilic elements.

Molecular transducers 12-1, 12-2, and 12-3 are also dispersed within the protective layer 11, and extend beyond it, into the sensing environment 56. In this illustration (FIG. 5B, step 3), two types of molecular transducers are provided; one type is shown as 12-1 and 12-2, the other type is shown as 12-3. In other arrangements, there may be only one kind of molecular transducer or there may be any number of kinds of molecular transducers provided in the protective layer 11. The molecular transducers provide communication through the protective layer 11 between the nanostructure 10 and the environment 56. The molecular transducers contain receptors that bind to specific molecules of interest from the environment 56, as was discussed in more detail above with reference to FIGS. 3 and 4. In sum, Step 3 provides functionalization for the nanostructure 10, thus forming a nano-sensor device 9.

In other embodiments, the nanostructure 10 can be formed in physical contact with the surface of the semiconductor chip in the region between the electrodes 32, as shown in the structure of FIG. 2A. The nanostructure can be formed in a groove in the semiconductor substrate, as shown in the structure of FIG. 2B. The nanostructure can be formed and then attached to the electrodes of the semiconductor chip, or the nanostructure can be formed on the chip from electrode to electrode. Subsequently the biologically and chemically protective coating or layer containing molecular transducers is formed over at least part of the nanostructure as it lies on the semiconductor chip. In other embodiments the nanostructure can span across three electrodes, thus forming two nanostructure regions. One region can be functionalized as described above. The second region can be functionalized and then passivated to be used as a control region. Differences in current flow between one region and the other can indicate the sensing of target molecules in the non-passivated region.

The advantages of biological agent detection using an electrical sensor with control circuitry integrated on the same chip are significant. These advantages include high speed of detection, low power requirements, small size, and low manufacturing costs because of the use of standard manufacturing technologies. Advantages also include high sensitivity, for example, of the order of a part-per-billion (ppb) without any need to increase the quantity of the sample material. Taken together, these advantages contribute to an enabling of remote sensing, data transmission, and ultimately a large physical area to be pervasively and effectively monitored for the presence of human pathogens.

This invention has been described herein in sufficient detail to provide those skilled in the art with the information needed to apply the novel principles and to construct and use such specialized components as are required. However, it is understood that the invention can be carried out by different equipment and devices, and that various modifications, both as to the equipment and operating procedures, can be accomplished without departing from the scope of the invention itself.

We claim:

1. A nanostructure sensing device for an analyte, including:
a least one nanostructure;
a layer encapsulating at least a portion of the nanostructure, the layer configured to inhibit non-specific interaction between the nanostructure and molecules other than the analyte; and
at least one molecular transducer configured to interact with the analyte, the transducer connected to the nanostructure and extending therefrom into a local environment.

2. The device of claim 1, wherein the analyte is a biomolecule.

3. The device of claim 2, wherein the biomolecule is.diagnostic of a predetermined human pathogenic organism.

4. The device of claim 3, wherein the biomolecule is selected from a group including a peptide, a protein, a glycoprotein, a lipid, a glycolipid, a carbohydrate, a polysaccharide, a polynucleotide sequence, and a molecule of intermediary metabolism.

5. The device of claim 1, wherein the layer includes molecules selected from the group consisting of amphipathic molecules configured into a monolayer; a bilayer; a multilayer; a micellar layer; or hybrid bilayer; a self-assembled monolayer of biological materials, a self-assembled monolayer of non-biological materials; a polymer layer; or other amphipathic macromolecular assemblies.

6. The device of claim 1, wherein the nanostructure is selected from the group consisting of nanofibers, single-walled nanotubes, multi-walled nanotubes, nanocages, nanococoons, nanohorns, nanotopes, nanotori, nanorods, nanowires, and other fullerene-like molecules.

7. The device of claim 1, wherein the molecular transducer includes:
a receptor molecule configured to bind to the analyte and upon such binding, change in molecular configuration, such change representing a signal; and
an anchor molecule functionally connected to the receptor molecule and configured to modify a conductivity of the nanostructure upon receipt of the signal from the receptor.

8. The devise of claim 1, wherein the nanostructure has a conductivity, the nanostructure configured to change said conductivity upon receipt of a signal from the molecular transducer.

9. The devise of claim 8, further including:
a signal processing unit configured to provide analysis of the changes in conductivity of the nanostructure;
a record keeping device for storage of the analysis of changes in conductivity; and
an alarm configured to respond upon receipt of conductivity changes that exceed a predetermined threshold.

10. The device of claim 7, wherein the receptor molecule is selected from a group that includes an antibody, a portion of an antibody, an enzyme, a portion of an enzyme, a DNA sequence, an RNA sequence, a carbohydrate, a cyclodextrin, a crown ether, or any other molecule capable of specifically recognizing and binding the analyte.

11. The device of claim 7 further including a tether molecule functionally interposed between the receptor molecule and the anchor molecule.

12. An electronic sensing system including:
a substrate;
at least one nanostructure with a layer encapsulating at least a portion thereof, the nanostructure having a conductivity, the encapsulated nanostructure exposed to a local environment;
a first conductive element disposed over the substrate and making a first electrical connection to the nanostructure;
a second conductive element disposed over the substrate and making a second electrical connection to the nanostructure; and
at least one molecular transducer configured to interact with a biomolecular analyte, the transducer attached to the nanostructure and extending therefrom into the local environment;
wherein the molecular transducer is also configured to modify the conductivity of the nanostructure upon interaction with the analyte.

13. A circuit for detecting an analyte diagnostic of a pathogenic agent, including:
an electrical supply;
a meter connected to the electrical supply;
a substrate;
at least one nanostructure disposed over the substrate and exposed to a local environment, the nanostructure having a conductivity;
a layer encapsulating at least a portion of the nanostructure;
a first conductive element disposed over the substrate and making a first electrical connection to the nanostructure;
a second conductive element disposed over the substrate and making a second electrical connection to the nanostructure; and
at least one molecular transducer configured to interact with the analyte, the transducer anchored to the nanostructure and extending therefrom into the local environment;
wherein the molecular transducer is also configured to modify the conductivity of the nanostructure upon interaction with the analyte; and
wherein the first and second conductive elements are in electrical connection with the electrical supply and the meter.

14. The circuit of claim 13, wherein the substrate is configured to function as a gate electrode within the circuit.

15. A sensing device for a biomolecular analyte, including:
a substrate;
a least one nanostructure disposed over the substrate, the nanostructure having a conductivity;
a layer encapsulating at least a portion of the nanostructure;
an electrical circuit connected to the nanostructure, configured to detect changes in conductivity; and
at least one molecular transducer configured to interact with the analyte, the transducer attached to the nanostructure and extending therefrom into a local environment;
wherein the molecular transducer is also configured to modify the conductivity of the nanostructure upon interaction with the analyte.

16. The sensing device of claim 15, wherein the nanostructure is selected from the group consisting of nanofibers, single-walled nanotubes, multi-walled nanotubes, nanocages, nanococoons, nanohorns, nanotopes, nanotori, nanorods, nanowires, and other fullerene-like molecules.

17. The sensing device of claim 15, wherein the layer is selected from the group consisting of amphipathic molecules configured into a monolayer; a bilayer; a multilayer; a micellar layer; or hybrid bilayer; a self-assembled monolayer of biological materials, a self-assembled monolayer of non-biological materials; a polymer layer; or other amphipathic macromolecular assemblies.

18. The sensing device of claim 17, wherein the layer includes polyethylene glycol.

19. The sensing device of claim 15, wherein the molecular transducer includes:
a receptor molecule configured to bind to the analyte and upon binding, to change in molecular configuration;
an anchor molecule configured to modify the conductivity of the nanostructure upon the change in molecular configuration of the receptor molecule; and
a tether molecule connecting the receptor molecule and the anchor molecule.

20. A biomolecular analyte sensing device, including:
a substrate;
a plurality of nanostructures disposed over the substrate, each nanostructure having a conductivity;
a first conductive element disposed over the substrate and connected to each nanostructure;
a second conductive element disposed over the substrate and connected to each nanostructure;
a layer encapsulating at least a portion of each of the nanostructures; and
a plurality of molecular transducers configured to interact with a biomolecular analyte, the transducers attached to the nanostructures and extending therefrom through the layer and into a local environment;
wherein, the molecular transducers, upon interaction with the analyte in the local environment, are configured to modify the conductivity of the nanostructure to which they are attached.

21. The sensing device of claim 21, wherein the plurality of molecular transducers includes more than one type of transducer, the transducers types being different by being configured to interact with different analytes.

22. An electronic circuit for detecting a biomolecular analyte, including:
at least one nanostructure disposed over a substrate; the nanostructure having a conductivity;
a layer encapsulating at least a portion of the nanostructure;
at least one molecular transducer attached to the nanostructure and extending therefrom through the layer; the transducer configured both to bind a biomolecular analyte and to attach to the nanostructure, the transducer further configured to undergo a change in molecular configuration upon binding of the analyte;.
at least two electrodes electrically connected to the nanostructure;
a power supply configured to apply a voltage to the electrodes; and
an electrical meter configured to measure the current conducted across the circuit upon application of voltage.

23. A method of making a nanostructure sensing device for a biomolecular analyte, including the steps of:
disposing at least one nanostructure over a substrate, the nanostructure have a conductivity;
encapsulating at least a portion of the nanostructure with a coating;
attaching a molecular transducer to the nanostructure, the transducer configured to bind the biomolecular analyte and further configured to modify the conductivity of the nanostructure upon binding of the analyte;
forming a first conductive element connected to the nanostructure; and
forming a second conductive element connected to the nanostructure.

24. A method of sensing a biomolecular analyte with a nanostructure sensing device, including the steps of:
binding the analyte to a receptor, thereby inducing a change in molecular configuration of the receptor;
transmitting a force generated by the change in the configuration of the receptor to an anchor molecule, the anchor molecule connected both to the receptor and a nanostructure;
modifying the interaction between the anchor molecule and the nanostructure as a consequence of the force exerted upon it by the change in the configuration of the receptor molecule; and
changing the conductivity of the nanostructure as a consequence of the modifying of the interaction between the anchor molecule and the nanostructure.

25. The method of claim 24, where the modified interaction between the anchor molecule and the nanostructure includes a structural deformation of the nanostructure.

26. The method of claim 24, wherein the modified interaction between the anchor molecule and the nanostructure includes electrical modifications of a type selected from a group including charge transfer, change in the local electric field, change in the distortion of bonds, and change in the π-bond overlaps.

27. A method of sensing an analyte diagnostic of a pathogenic agent, including the steps of:
providing a nanostructure sensing device having at least one nanostructure in contact with at least two electrodes; the nanostructure having a conductivity, the nanostructure having a molecular transducer attached to it, the nanostructure further being at least partially covered with a coating;
measuring a first electrical signal under control conditions, in the absence of the analyte;
binding the analyte to a receptor, thereby inducing a change in molecular configuration of the receptor;
transmitting a force generated by the change in the configuration of the receptor to an anchor molecule, the anchor molecule connected both to the receptor and the nanostructure;
modifying the interaction between the anchor molecule and the nanostructure as a consequence of the force exerted upon it by the change in the configuration of the receptor molecule;
changing the conductivity of the nanostructure as a consequence of the modifying of the interaction between the anchor molecule and the nanostructure;
measuring a second electrical signal after exposing the device to a test sample, possibly containing the analyte; and
correlating a change between the first electrical signal and the second electrical signal to the sensing of the analyte.
